(19) United States
(12) Patent Application Publication
(10) Pub. No.: US 2024/0420822 A1
(43) Pub. Date: Dec. 19, 2024

(54) Behavior Enhancement and Goal-Setting System and Sleeve Arrangement

(71) Applicant: Anthony L. Clarno, Brookfield, IL (US)

(72) Inventor: Anthony L. Clarno, Brookfield, IL (US)

(21) Appl. No.: 18/822,346

(22) Filed: Sep. 2, 2024

Related U.S. Application Data
(63) Continuation-in-part of application No. 16/397,876, filed on Apr. 29, 2019, now Pat. No. 12,075,867.

(51) Int. Cl. G16H 20/30; A41D 13/00

(52) U.S. Cl. CPC G16H 20/30; A41D 13/0015

(57) ABSTRACT

A behavior enhancement and goal-setting system and sleeve arrangement provides a user with a physical representation of the user's progress in an activity so as to reinforce behavior supportive of the activity. The basic behavior enhancement and goal-setting sleeve arrangement includes a sleeve and an array of activity reinforcement badges. The sleeve is formed from an elastic fabric material and includes proximal and distal open ends, and inner and outer sleeve surfaces. Activity reinforcement badges are attachable to the outer sleeve surface marked with a periodic tiling pattern including an array of tiles configured to visually demarcate an array of activity reinforcement badge attachment sites. The badges are sized and shaped to correspond to a tile size and a tile shape within the array of tiles.

### PRIOR HISTORY

This is a Continuation-in-Part patent application claiming the benefit of U.S. patent application Ser. No. 16/397,876 filed in the United States Patent and Trademark Office on 29 Apr. 2019.

### TECHNOLOGICAL FIELD

The presently disclosed subject matter relates generally to a behavior enhancement and goal-setting system involving wearable fabrics and benchmark badges attachable thereto. More particularly, the presently disclosed subject matter relates to a sleeve arrangement wearable by a user that may be outfitted with benchmark badges designed to create a physical representation of a participant's progress or participation in any activity or sport with a predetermined curriculum and/or activity experience so as to reinforce positive behavior and skill development in the activity or sport.

### BRIEF DESCRIPTION OF THE PRIOR ART

For many years it has been documented that there is a real benefit to creating reward systems in education and activities for children and adults. For example, the various scouting programs in America use badges, sewn onto a uniform, to denote accomplishments inside of their unique scouting programs. To explain this more specifically; as a child participates in and completes an activity a “Merit Badge” might be bestowed upon the child as a symbol for their having completed a task or learned a new skill. The badge and symbol constitute a reward for the accomplishment, which then inspires the child to learn more material to earn more badges.

Many schools are known to create “Gold Star” programs, or some variation thereof, which allow children to be recognized for academic or behavioral accomplishments in the classroom or institution with a gold star sticker or stamp. It has shown great success in generating motivation and holding children accountable for their choices. What's more, it allows parents and program administrators to hold professionals or volunteers accountable for the progress and inspiration of the children they are teaching.

Both the “Gold Star” and “Merit Badge” programs are very general examples of reward systems in the field of childhood education. While effective, neither of them was specifically created with the child athlete in mind. These reward programs were developed to be utilized in either the classroom or in combination with a uniform or sash that would not function safely in a high impact or fast paced activity like gymnastics, basketball or the martial arts.

For example, a child or person performing a summersault would not be safe wearing the equivalent of a Girl Scout's sash because it is not form-fitting. It could become tangled and potentially cause injury during the exercise. The prior art thus perceives a need for an activity-reinforcing wearable fabric article that is both form-fitting and does not become tangled or create the potential for injury during target activity or behavior development as summarized in more detail hereinafter.

It is the intention of the Achieve Sleeve activity-reinforcing sleeve arrangement according to the presently disclosed subject matter described more fully hereinafter to recognize and encourage accomplishments by a participant for any activity or experience while also remaining safe to wear during the activity itself, regardless of the level of physicality.

### GENERAL DESCRIPTION

Among the many objectives of the presently disclosed subject matter is the provision of a behavior enhancement and goal-setting article or sleeve arrangement for providing a user with a physical representation of the user's progress in an activity with a predetermined curriculum so as to reinforce behavior supportive of the activity. The behavior enhancement and goal-setting sleeve arrangement according to the presently disclosed subject matter may be said to essentially comprise a sleeve, and an array of activity reinforcement badges usable within a system for rewarding participant activity therewithin.

The sleeve or sleeve-like article is preferably formed from form-fitting fabric material having certain elasticity, and preferably comprises a proximal open end, a distal open end, an anterior side, a posterior side, a sleeve long axis, an inner sleeve surface, and an outer sleeve surface. The outer sleeve surface primarily receives the activity reinforcement badges, and the inner sleeve surfacing primarily opposes or engages a user's anatomical or skin surface as at the user's forearm or lower leg.

The foundation badge is preferably attached to the outer sleeve surface at the posterior side and proximal open end, and comprises a proximal foundation badge edge and a distal foundation badge edge. The distal foundation badge edge comprises an array of primary activity badge alignment demarcations that aligned end-to-end along the distal foundation badge edge for demarcating an array of activity badge alignment regions upon the outer sleeve surface in distal adjacency to the distal foundation badge edge.

The activity reinforcement badges according to the presently disclosed subject matter, including the foundation or reference point badge, the primary activity badges, and the secondary activity badges, are preferably adhesively attachable to the outer sleeve surface. The behavior enhancement and goal setting apparatus according to the presently disclosed subject matter further contemplates that at least one primary activity badge is preferably attachable to the outer sleeve surface at an activity badge alignment region in distal adjacency to the distal foundation badge edge.

The primary activity badge comprises a proximal primary activity badge edge and a distal primary activity badge edge. The primary activity badge further preferably comprises a foundation badge alignment demarcation at or adjacent the proximal primary activity badge edge. The foundation badge alignment demarcations help the user or participant align the primary activity badges with the foundation badge at or adjacent the distal foundation badge edge.

The distal foundation badge edge is preferably arcuate, the array of primary activity badge alignment demarcations each comprise a foundation badge demarcation arc length. The foundation badge alignment demarcation at the proximal primary activity badge edge comprises a primary activity badge demarcation arc length such that the primary activity badge demarcation arc lengths are greater in magnitude than the foundation badge demarcation arc lengths for aiding the user in aligning the primary activity badge in spaced relation to the distal foundation badge edge.

The array of primary activity badge alignment demarcations may preferably comprise primary activity badge-matching coloration, and the primary activity badges may preferably comprise foundation badge demarcation-matching coloration. The foundation badge demarcation-matching coloration aids the user or participant in aligning the primary activity badges in adjacency to corresponding portions of the foundation badge via a color-matched badge alignment demarcation pairing comprising corresponding primary activity badge alignment demarcations and foundation badge alignment demarcations. The color-matched badge alignment demarcation pairing may further comprise a first color shade, and each primary activity badge may further preferably comprise a second color shade distal to the foundation badge alignment demarcation within a symbol-bearing region of each primary activity badge.

The behavior enhancement and goal setting apparatus according to the presently disclosed subject matter may further preferably comprise an array of bonus or secondary activity badges. Each secondary activity badge is also attachable to the outer sleeve surface in distal adjacency to the array of activity badge alignment regions at the posterior side and in proximal adjacency to the distal open end. The primary and secondary activity badges may both preferably comprise activity symbol markings that generally coincide with the activity that is to be reinforced.

There is provided in accordance with another aspect of the presently disclosed subject matter a behavior enhancement and goal-setting system for providing a user with a wearable physical representation of the user's progress in an activity so as to reinforce behavior supportive of the activity. The behavior enhancement and goal-setting system comprising at least one wearable fabric article and at least one activity reinforcement badge. The wearable fabric article has an outer article surface, which outer article surface is marked with a periodic tiling pattern comprising an array of tiles configured to visually demarcate an array of activity reinforcement badge attachment sites.

The activity reinforcement badges are configured for attachment to a select activity reinforcement badge attachment site of the array of activity reinforcement badge attachment sites. Each activity reinforcement badge is sized and shaped to correspond to a tile size and a tile shape within the array of tiles. In some embodiments, the wearable fabric article is characterized by a sleeve, the sleeve comprising a proximal open end, a distal open end, an outer sleeve surface defining the outer article surface and a sleeve length extending along a sleeve long axis. In some embodiments, the periodic tiling pattern is a regular periodic tiling pattern comprising an array of regular shapes.

In some embodiments, the array of regular shapes is defined by shape-defining grid pattern, the shape-defining grid pattern comprising a uniform tile-to-tile thickness. In some embodiments, the regular periodic tiling pattern is a hexagonal tiling pattern. In some embodiments, the hexagonal tiling pattern comprises an array of regular hexagonal shapes each of which comprise a long diagonal axis. In some embodiments, the long diagonal axes extend widthwise across the outer article surface in parallel relation to the proximal open end and distal open end transverse to the sleeve long axis.

In some embodiments, the outer article surface comprises a color code band portion, the color code band portion comprising a color code for denoting a participant activity level. In some embodiments, the activity reinforcement badge comprises a color code badge portion corresponding to the color code band portion. In some embodiments, the color code band portion is proximally located, distally extending from the proximal open end a band thickness.

In some embodiments, the system is based on a first sleeve and at least one second sleeve. The first sleeve comprises a first color code band portion, the first color code band portion for denoting a first set of curricula associated with a first color. The at least one second sleeve comprises a second color code band portion, the second color code band portion for denoting a second set of curricula associated with a second color. The at least one second sleeve may be awarded to the user upon the user's completion of the first set of curricula.

### BRIEF DESCRIPTIONS OF THE DRAWINGS

Other features and objectives of the invention will become more evident from a consideration of the following brief descriptions of patent drawings.

FIG. 1 is a perspective view of a behavior enhancement and goal-setting long sleeve arrangement according to the presently disclosed subject matter.

FIG. 1A is an enlarged plan view of a central foundation or reference point badge according to the presently disclosed subject matter surrounded by an array of activity badge alignment regions for placing/positioning first activity badges according to the presently disclosed subject matter.

FIG. 1B is an enlarged plan view of a central foundation or reference point badge surrounded by an array of first activity badges according to the presently disclosed subject matter.

FIG. 2 is a perspective view of a participant wearing a behavior enhancement and goal-setting long sleeve arrangement according to the presently disclosed subject matter with an instructor's hand adhesively applying a second activity badge to an outer sleeve surface of a long sleeve of the behavior enhancement and goal-setting long sleeve arrangement.

FIG. 3 is a perspective view of an adhesive strip of second activity badges according to the presently disclosed subject matter with a bottom second activity badge being peeled from the adhesive strip.

FIG. 4 is a first sequential perspective view of a long sleeve according to the presently disclosed subject matter devoid of all activity badges prior to application thereof to the long sleeve.

FIG. 5 is a second sequential perspective view of the long sleeve otherwise depicted in FIG. 4 showing a foundation or reference point badge firstly attached to the outer sleeve surface adjacent the proximal open end, and a first activity badge attached to the outer sleeve surface adjacent the distal open end thereby providing a long sleeve arrangement according to the presently disclosed subject matter.

FIG. 6 is a third sequential perspective view of the long sleeve arrangement otherwise depicted in FIG. 5 showing a first activity badge newly attached to the outer sleeve surface in adjacency to the foundation or reference point badge; and a second activity badge attached to the outer sleeve surface adjacent the distal open end.

FIG. 7 is a fourth sequential perspective view of the long sleeve arrangement otherwise depicted in FIG. 6 showing a second activity badge newly attached in adjacency to the foundation or reference point badge adjacent the firstly attached activity badge; and a third activity badge attached to the outer sleeve surface adjacent the distal open end.

FIG. 8 is a fifth sequential perspective view of the long sleeve arrangement otherwise depicted in FIG. 7 showing a third activity badge newly attached in adjacency to the foundation or reference point badge adjacent the secondly attached activity badge; and a fourth activity badge attached to the outer sleeve surface adjacent the distal open end.

FIG. 9 is a sixth sequential perspective view of the long sleeve arrangement otherwise depicted in FIG. 8 showing a fourth activity badge newly attached in adjacency to the foundation or reference point badge adjacent the thirdly attached activity badge; and fifth and sixth activity badges attached to the outer sleeve surface adjacent the distal open end.

FIG. 10 is a perspective view of a behavior enhancement and goal-setting long sleeve arrangement according to the presently disclosed subject matter showing a foundation or reference point badge adjacent the proximal open end; an array of four activity badges adjacent the reference point badge; and an array of six activity badges adjacent the distal open end.

FIG. 11 is a distal end perspective view of the behavior enhancement and goal-setting long sleeve arrangement otherwise depicted in FIG. 10.

FIG. 12 is a perspective view of the behavior enhancement and goal-setting long sleeve arrangement otherwise depicted in FIGS. 10 and 11 worn by a participant upon the participant's forearm.

FIG. 13 is a perspective view of a long sleeve according to the presently disclosed subject matter as worn by a participant upon the participant's forearm with another person's hands actuating the proximal and distal open ends to demonstrate elasticity of the proximal and distal open ends of the long sleeve.

FIG. 14 is a perspective view of a long sleeve in a relaxed state as removed from a participant's arm to demonstrate flexibility of the fabric material construction.

FIG. 15 is a perspective view of a short sleeve according to the presently disclosed subject matter as worn by a participant upon the participant's lower leg with another person's hand actuating the region intermediate the proximal and distal open ends to demonstrate elasticity of the region intermediate the proximal and distal open ends of the short sleeve.

FIG. 16 is a distal end perspective view of a short sleeve arrangement according to the presently disclosed subject matter showing the short sleeve outfitted with an array of three activity badges upon the outer sleeve surface.

FIG. 17 is a perspective view of the short sleeve arrangement according to the presently disclosed subject matter showing the short sleeve outfitted with an array of three activity badges upon the outer sleeve surface.

FIG. 18 is a side view depiction of the short sleeve arrangement according to the presently disclosed subject matter as worn by a participant at the participant's lower leg/ankle region.

FIG. 19 is a side view depiction of the short sleeve arrangement according to the presently disclosed subject matter as worn by a participant at the participant's wrist region.

FIG. 20 is a tabular depiction of an array of four activity badges according to the presently disclosed subject matter alongside textual primary skills associated with the four activity badges.

FIG. 21 is a tabular depiction of an array of six activity badges according to the presently disclosed subject matter respectively depicting textual categories associated with the six activity badges.

FIG. 22A is a diagrammatic depiction of a transverse cross-section of a sleeve according to the presently disclosed subject matter depicting the sleeve in a relaxed state with a relaxed sleeve diameter.

FIG. 22B is a diagrammatic depiction of a transverse cross-section of a participant's forearm depicting a relatively larger diameter of the participant's forearm as compared to the relaxed sleeve diameter otherwise depicted in FIG. 22A.

FIG. 22C is a diagrammatic depiction of a transverse cross-section of a participant's forearm depicting the diameter of the participant's forearm on par with an actuated sleeve diameter once worn upon the forearm of the participant.

FIG. 23 is an enlarged, fragmentary diagrammatic depiction of a section of sleeve depicting an upper, outer material layer and a lower, inner material layer.

FIG. 24A is an enlarged edge view depiction of a foundation or reference point badge according to the presently disclosed subject matter showing an upper, outer layer and a lower, inner layer.

FIG. 24B is an enlarged edge view depiction of a first activity badge according to the presently disclosed subject matter showing an upper, outer layer and a lower, inner layer.

FIG. 24C is an enlarged edge view depiction of a second activity badge according to the presently disclosed subject matter showing an upper, outer layer and a lower, inner layer.

FIG. 25A is a first tabular depiction of an array of three exemplary activity curricula alongside activity badges associated therewith.

FIG. 25B is a second tabular depiction of an exemplary primary activity curriculum alongside an activity badge associated therewith and an exemplary secondary activity curriculum.

FIG. 26 is an enlarged plan view of a central foundation or reference point badge according to the presently disclosed subject matter depicting arc lengths of primary activity badge alignment demarcations as juxtaposed adjacent foundation badge alignment demarcations with relative arc lengths thereof being further depicted.

FIG. 27 is an enlarged plan view of a central foundation or reference point badge according to the presently disclosed subject matter with an array of activity badges positioned or aligned in adjacency thereto and depicting color-coded pairing between the primary activity badge alignment demarcations of the foundation badge and the foundation badge alignment demarcations of the activity badges.

FIG. 28 is an enlarged, fragmentary depiction of proximal portions of a behavior enhancement and goal-setting long sleeve arrangement according to the presently disclosed subject matter showing an upper boundary edge outfitted with first coloration to depict an entry level behavior enhancement and goal-setting long sleeve arrangement.

FIG. 29 is an enlarged, fragmentary depiction of proximal portions of the behavior enhancement and goal-setting long sleeve arrangement according to the presently disclosed subject matter showing an upper boundary edge outfitted with second coloration to depict an advanced level behavior enhancement and goal-setting long sleeve arrangement.

FIG. 30 is an enlarged, fragmentary sectional view as enlarged and sectioned from FIG. 26 to more clearly depict the juxtaposition of a primary activity badge alignment demarcation and a foundation badge alignment demarcation according to the presently disclosed subject matter.

FIG. 31 is an enlarged diagrammatic depiction of a regular hexagon shape or tile pattern depicting a long diagonal axis, and edge length, and an incircle radius, the long diagonal axis being vertically oriented on the page.

FIG. 32 is an enlarged view of an upper, outer surface of an activity badge depicting a character image aligned along a vertically extending long diagonal axis of the activity badge formed in the shape of a regular hexagon.

FIG. 33 is an enlarged view of an upper, outer surface of an activity badge depicting a character image aligned along a horizontally extending long diagonal axis of the activity badge formed in the shape of a regular hexagon.

FIG. 34 is an enlarged elevational view of a short sleeve according to the presently disclosed subject matter depicting a tessellated outer sleeve surface showing regular hexagonal patterns arranged in linear rows such that the long diagonal axes of the hexagonal tiles extend lengthwise intermediate the proximal open end and the distal open end in parallel to the sleeve long axis.

FIG. 35 is an enlarged elevational view of a short sleeve according to the presently disclosed subject matter depicting a tessellated outer sleeve surface showing regular hexagonal patterns arranged in linear rows such that the long diagonal axes of the hexagonal tiles extend widthwise intermediate the proximal open end and the distal open end transversely relative to the sleeve long axis.

FIG. 36 is an enlarged elevational view of a long sleeve according to the presently disclosed subject matter depicting a tessellated outer sleeve surface showing regular hexagonal patterns arranged in linear rows such that the long diagonal axes of the hexagonal tiles extend widthwise intermediate the proximal open end and the distal open end transversely relative to the sleeve long axis.

FIG. 37 is an enlarged elevational view of a long sleeve according to the presently disclosed subject matter depicting a tessellated outer sleeve surface distally located relative to a proximally located band free of tessellation.

FIG. 38 is an enlarged elevational view of a long sleeve according to the presently disclosed subject matter depicting a tessellated outer sleeve surface distally located relative to a proximally located color code band free of tessellation having a first coloration.

FIG. 39 is an enlarged elevational view of a long sleeve according to the presently disclosed subject matter depicting a tessellated outer sleeve surface distally located relative to a proximally located color code band free of tessellation having a second coloration.

### DETAILED DESCRIPTION OF THE EMBODIMENTS

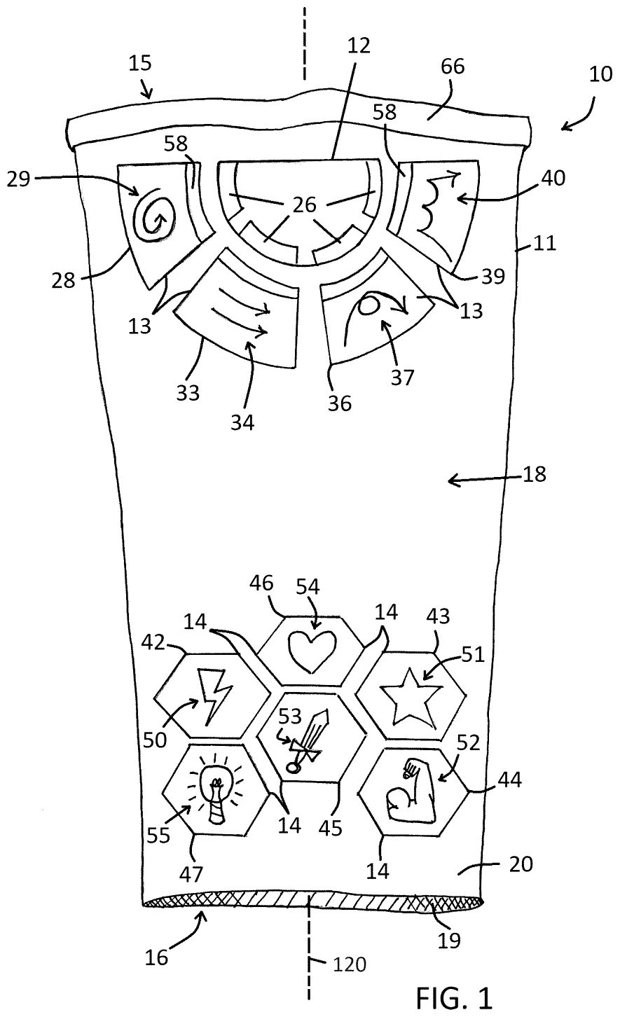

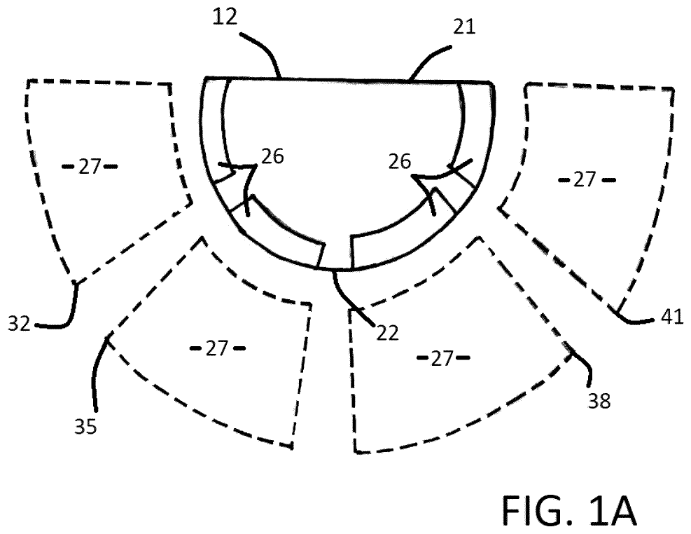

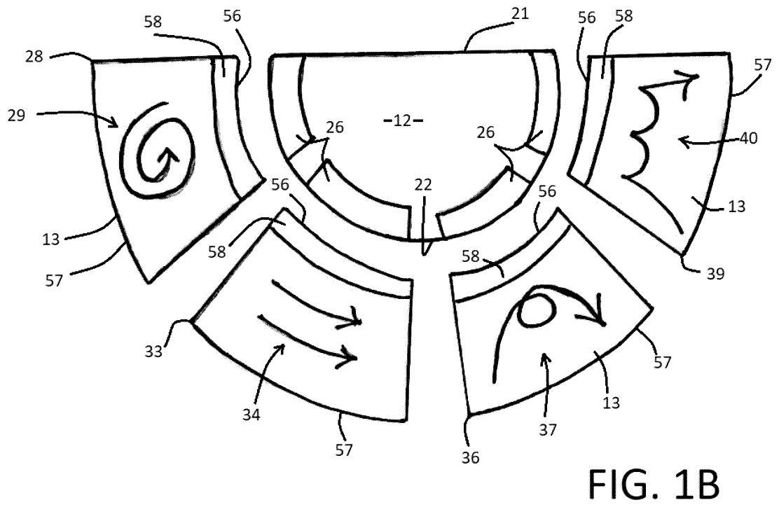

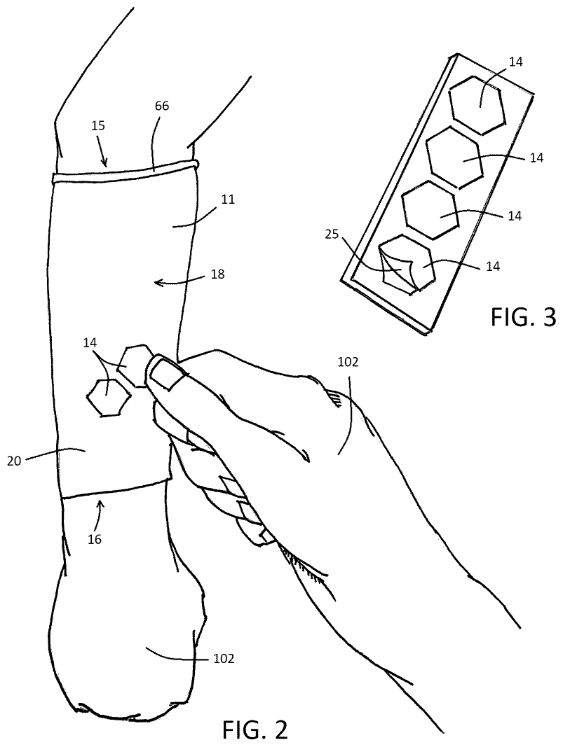

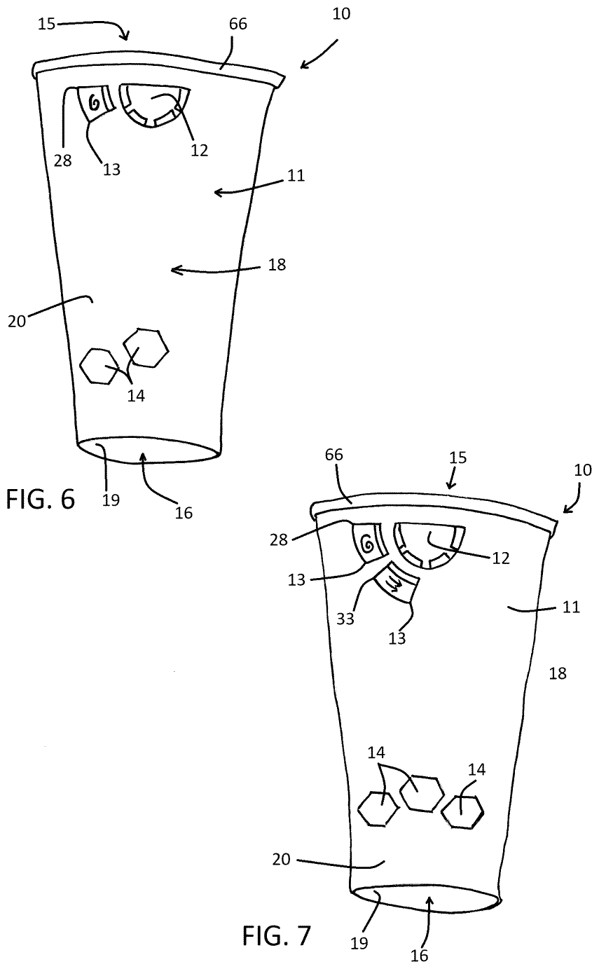

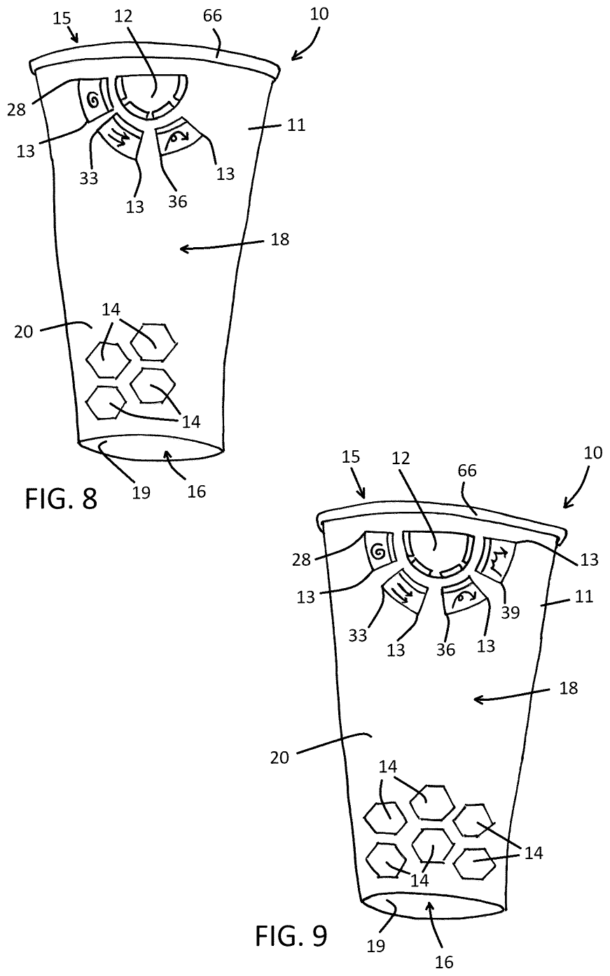

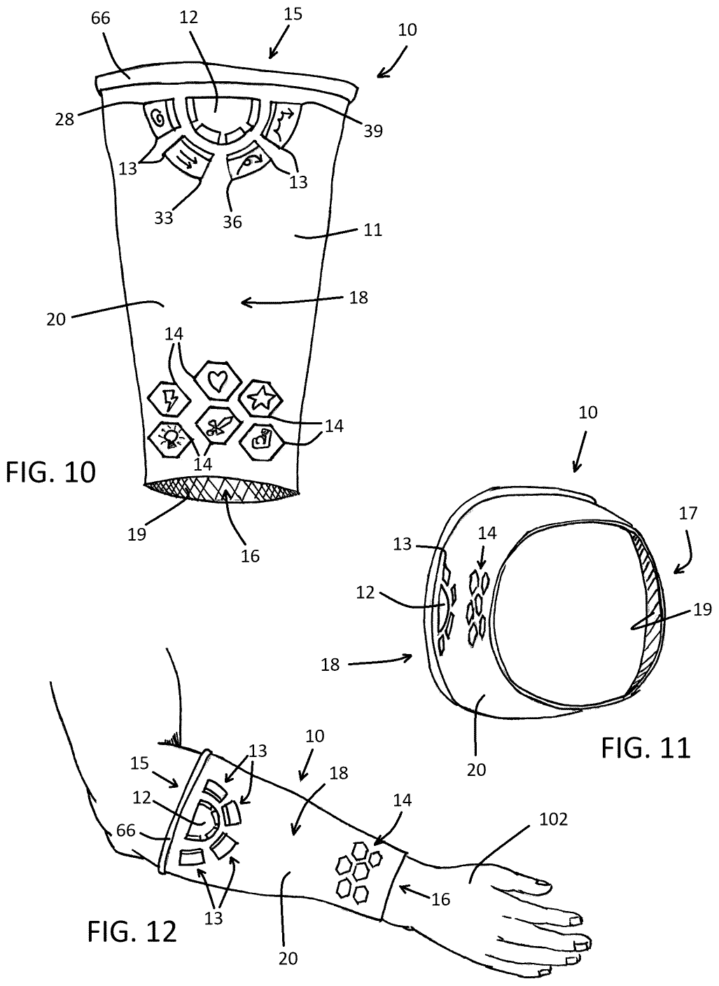

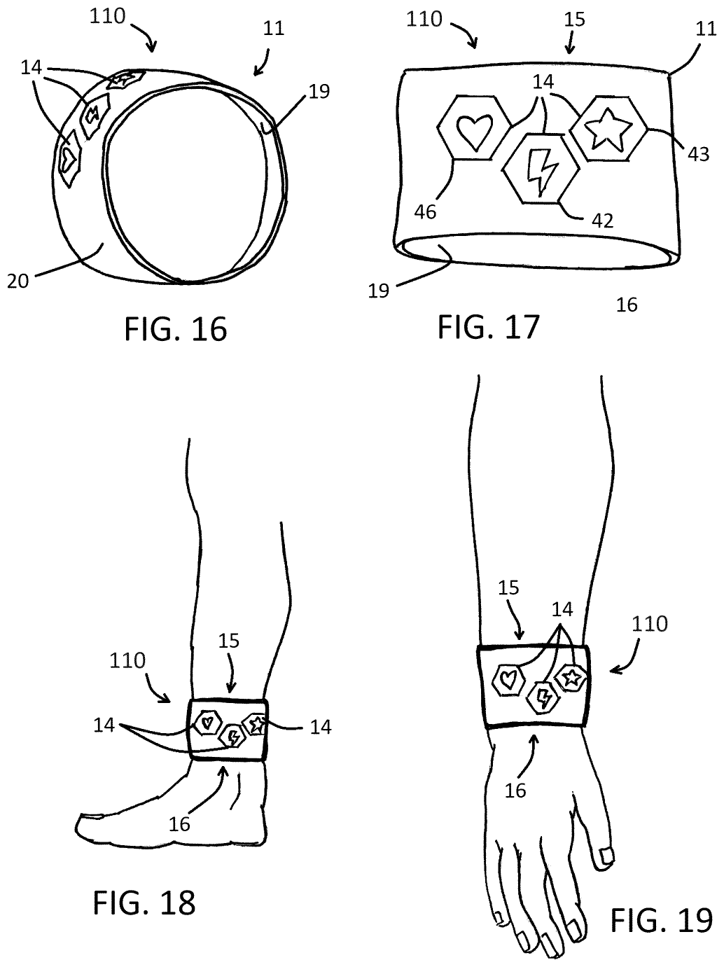

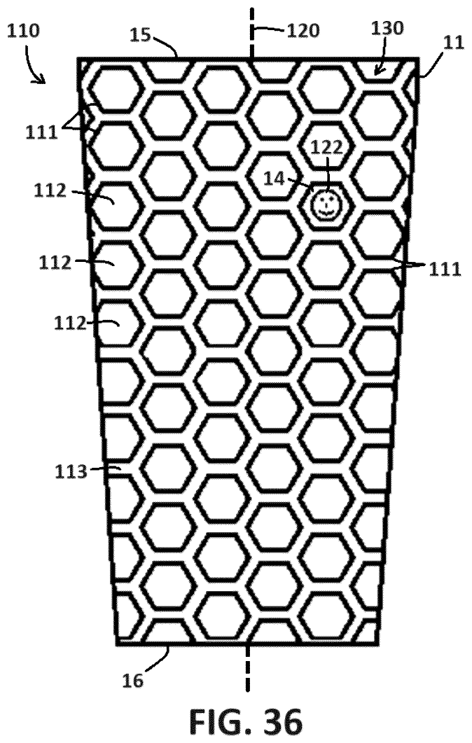

Referring now to the drawings with more specificity, the following specifications generally describe a behavior enhancement and goal-setting system and sleeve arrangement as variously exemplified. The behavior enhancement and goal-setting system is based on a wearable fabric article for providing a user or participant with a physical representation of the user's progress in an activity (e.g. gymnastics activity) having a predetermined curriculum or curricula as generally depicted and referenced at 92 so as to reinforce behavior in the activity. In some embodiments, the wearable fabric article may be exemplified by a sleeve as at 11. The sleeve 11 may be either a long sleeve as generally depicted in FIGS. 1, 2, 4-14, and 36-39 or a short sleeve as generally depicted in FIGS. 15-19, 34, and 35.

A first behavior enhancement and goal-setting sleeve arrangement 10 according to the presently disclosed subject matter be said to essentially comprise sleeve 11 and an array of activity or behavior reinforcement badges or benchmark badges as variously exemplified. In some embodiments, the activity or behavior reinforcement badges of the first behavior enhancement and goal-setting sleeve arrangement 10 may include at least one foundation or reference point badge as at 12, at least one primary activity badge as at 13, and at least one secondary activity badge as at 14. The first behavior enhancement and goal-setting sleeve arrangement 10 according to the presently disclosed subject matter comprises differently shaped benchmark badges 12, 13, and 14 in some embodiments.

A second behavior enhancement and goal-setting sleeve arrangement 110 according to the presently disclosed subject matter be said to essentially comprise sleeve 11 and an array of activity or behavior reinforcement badges or benchmark badges as exemplified by activity badges 14, which, in some embodiments, are hexagonally shaped. In other words, the second behavior enhancement and goal-setting sleeve arrangement 110 according to the presently disclosed subject matter eliminates the at least one foundation or reference point badge 12 and the at least one primary activity badge as at 13. The second behavior enhancement and goal-setting sleeve arrangement 110 comprises a singular type of benchmark badge exemplified by activity badges 14 in some embodiments.

In some embodiments, the behavior enhancement and goal-setting sleeve arrangement 10 according to the presently disclosed subject matter is built upon sleeve 11 or similar other sleeve-like article otherwise referred to as the “Achieve Sleeve” preferably created from the same form-fitting materials as a gymnastics leotard (cotton, spandex, nylon, etc.). The gymnastics leotard is a product specifically designed to keep athletes safe during the most challenging and dangerous of exercises, meaning it uses materials that are known to be safe and appropriate for the rigors of the sport. By using similar fabrics and design concepts the Achieve Sleeve or sleeve 11 is able to take advantage of the safety practices that have already become an industry standard in sporting activities as exemplified by gymnastics.

The preferred placement of the Achieve Sleeve on the participant's body derives from safety concerns. The sleeve 11 may, for example, be worn on the forearm between the wrist and elbow, making it an ideal location for product visibility while delivering no meaningful obstruction to the performance of any activity. By considering FIG. 1, the reader will note the sleeve 11 is formed so as to preferably conform to a forearm's uniquely conical shape. The distal open end 16 comprises a relatively narrow diameter adjacent the wrist and gradually widens out around the girth of the forearm. It can be made of any skin-engaging, appropriate materials that have flexibility so as to fit like a snug sock. Looseness may create the potential for distraction during exercises, or even inhibit the performance of skills and activities.

Conceivably, a multitude of fabric varieties and decorative elements may be used in the construction or formation of the sleeve 11. Accordingly, the sleeve 11 may be formed from inexpensive cotton or a higher quality hydrophobic, moisture-wicking fabric blend 91 while still achieving the intent of the product. In addition, the sleeve 11 may be outfitted with colorful bindings or fabric piping on either end, near the wrist or elbow or both, that advertise company colors or telegraph certain benchmarks in a program. A user could also add accoutrements, such as rhinestones or heat transfer logos, so long as the accoutrements do not inhibit the safety or effectiveness of the sleeve 11.

The sleeve 11 should be made of any fabric that allows for the adhesion of the uniquely designed Achieve Sleeve activity reinforcement or benchmark badges as described in more detail hereinafter. The activity reinforcement or benchmark badges according to the presently disclosed subject matter have been created to adhere to cotton or cotton blends for best effectiveness. In some embodiments, the sleeve 11 may be formed from a moisture-wicking, hydrophilic fabric material layer 90. The moisture-wicking, hydrophilic fabric material layer 90 may be at an outer layer or surface 20 of the sleeve 11 in some embodiments and may be at an inner layer or surface 19 of the sleeve 11 in some embodiments. In some embodiments, the sleeve may comprise a hydrophobic material layer opposite the hydrophilic material layer.

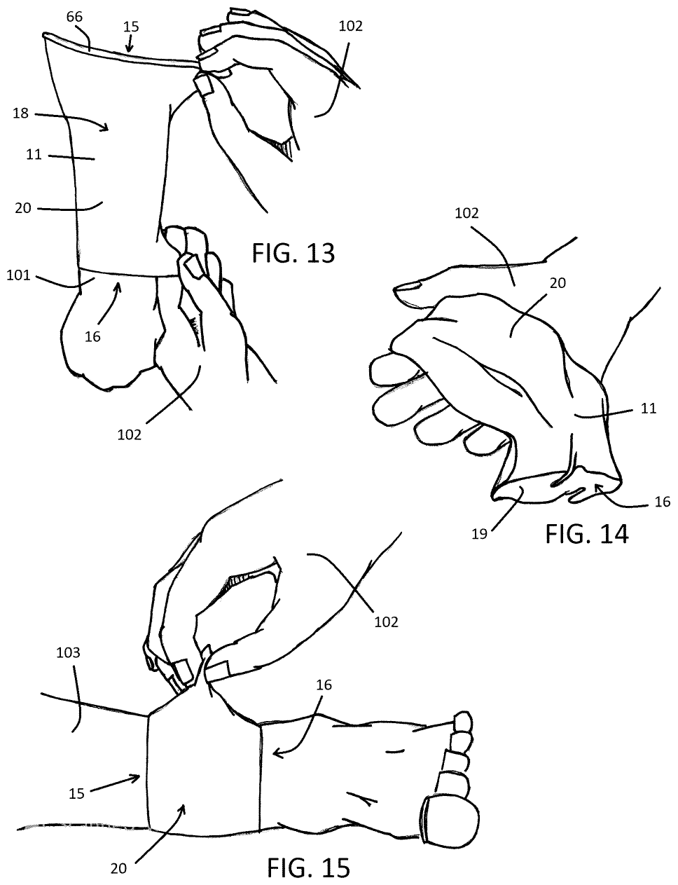

The sleeve 11 may be formed from a fabric material having a certain degree of form-fitting elasticity, in some embodiments. FIGS. 13-15 attempt to depict the sleeve 11 in various conditions of use. FIG. 13 depicts a sleeve 11 worn by the user upon the user's forearm as at 101 with a pair of hands 102 stretching the fabric material at the proximal open end 15 and distal open end 16 to depict elasticity of the fabric material at the ends 15/16. FIG. 14 attempts to depict the sleeve 11 removed from the user and being held aloft in a user's hand 102 in a relaxed state. FIG. 15 attempts to depict the sleeve 11 as worn upon a user's lower leg 103 with a hand stretching the fabric material intermediate the proximal open end 15 and the distal open end 16 to demonstrate elasticity of the fabric material at the region intermediate the proximal and distal open ends 15/16 as worn upon the user's lower leg 103.

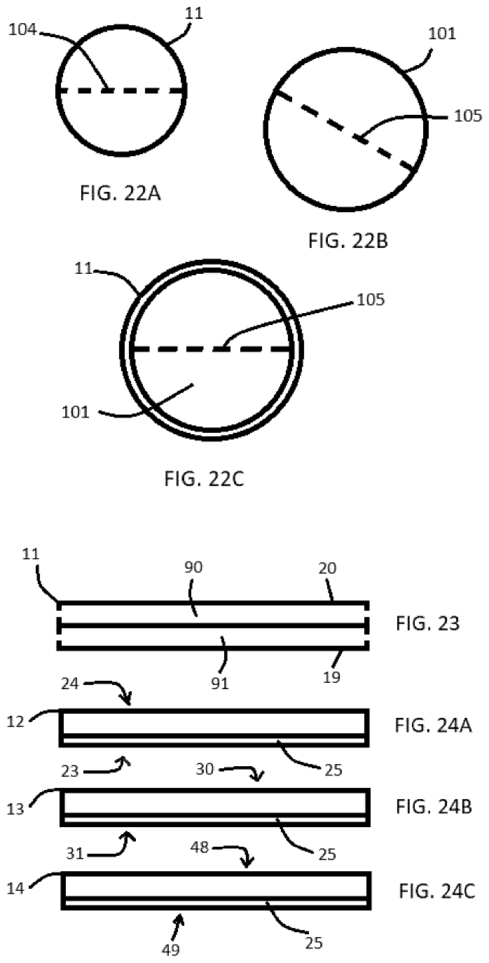

Comparatively referencing FIGS. 22A through 22B, the reader will there consider certain diagrammatic depictions of the sleeve 11 in both relaxed and actuated states to demonstrate an increase is sleeve diameter when in an actuated sleeve state. FIG. 22A depicts a transverse cross-section of the sleeve 11 when in a relaxed state having a relaxed sleeve diameter as at 104. FIG. 22B depicts a transverse cross-section of a user's forearm 101 having a forearm diameter as at 105 greater in magnitude relative to the relaxed sleeve diameter 104. When worn upon the user's forearm 101, the sleeve 11 has an actuated sleeve diameter on a par with the forearm diameter 105 and greater in magnitude than the relaxed sleeve diameter 104. It will thus be understood the fabric material preferably comprises certain elasticity for conforming the sleeve 11 to the user's anatomy (e.g. forearm 101 or lower leg 103) as worn for preventing axial displacement along the length of the user's anatomy.

The sleeve 11 preferably comprises a proximal open end as at 15; a distal open end as at 16; an anterior side as at 17; a posterior side as at 18; a sleeve long axis as at 120; an inner sleeve surface as at 19; and an outer sleeve surface as at 20. The outer sleeve surface 20 preferably comprises fabric surfacing conducive for adhesively attaching the activity or behavior reinforcement badges as variously exemplified by the foundation or reference point badge 12, and the primary and secondary activity badges 13 and 14, respectively. The inner sleeve surface 19 preferably comprises fabric surfacing conducive for opposing a user's skin surface, in some embodiments.

Similar to Boy Scout or Girl Scout badges, the activity reinforcement badges according to the presently disclosed subject matter otherwise referred to as Achieve Sleeve Badges are used to signify any accomplishment or completion of an activity. Once the participant has completed the task assigned or reached a benchmark in their curriculum, they are then given a Badge as a symbol of their progress. For example, a martial artist might receive a Badge for having technically perfected a kick in their school, or a summer camp attendee could be given a Badge for building a fire.

Notably, the Achieve Sleeve Badges can function independent of the sleeve 11 as well. They can be placed on any article of clothing or accessory created from the same cotton or synthetic blend fabric with the sole purpose of symbolizing progress or accomplishment in any activity. The purpose of the badge is to generate a feeling of pride, enthusiasm, or joy in the participant that they have accomplished a task to completion. It is known that receiving symbols, such as these badges, inspire participants to continue with the activity or think positively of their experience. It is the continued participation and positive feelings from patrons that will create a clear value to businesses using the Achieve Sleeve Badges in combination with the Achieve Sleeve.

The activity reinforcement badges or Achieve Sleeve Badges are preferably two-sided as generally depicted in FIGS. 24A-24C. On one side is a highly aggressive adhesive layer as at 25 that permanently adheres to fabrics with pressure from whomever is applying the Badge. There is no need for a heat source or sewing of any kind, it simply peels off a waxed paper backing and can be applied to a fabric surface in the orientation desired.

On the other side of the Badge is a printable fabric material, which can be designed and cut into any shape or size. In some embodiments, the benchmark badges may be cut into specific shapes that match or correspond to a tessellated surface pattern 111 marked or printed upon the outer sleeve surface 20. The printable fabric material is s printable in such a way so as to allow a user to design an image on a computer, then run the fabric through a printer that processes and applies an ink, in the design created, to the badge or fabric. Any color or design created can be totally unique to the patron. The design can signify steps accomplishments in a program, the completion of an activity, or even any form of branding appropriate to the business.

Manufacturing the benchmark badges follows a relatively simple process. It starts with sheets of blank badge material, which has a paper backing, the adhesive in the middle, and the printable fabric on the other side. The sheets are run through a printer to be inked with the predetermined design. Once the ink is done drying, the badges are then cut with a guided machine, leaving individual badges with a paper backing. More particularly, the benchmark badges may be formed from an ink printable fabric with paper backer and a fabric adhesive roll with paper backer. The fabric product is run through a large format printer to be inked with a specific design.

The cured printed fabric is laid flat and the paper backer is removed. The adhesive with a paper backer is pressed onto the printed fabric. The fabric paper backer is discarded and the adhesive backer is left on. The ensemble or layered product with printed fabric layer and adhesive with backer is run through a plotter which cuts the badges into shapes (e.g., hexagons). The final product is cut down into strips of benchmark badges based on client specifications. The benchmark badges are machine washable and permanently adhere to fabric.

It is the intent of this section to provide a hypothetical scenario, in which a teacher in any given program might use the benchmark badges in a very practical manner. By going through this scenario in detail it will become apparent that the process for application of the benchmark badge goes hand in hand with its creation. The benefit of positive reinforcement and acknowledging programming accomplishments is a corner stone for the value of the product and should be illuminated for complete understanding of functionality.

Upon entering an exemplary program, a child participant would receive an Achieve Sleeve in the color of their choosing. The entry level Achieve Sleeve might have a yellow binding at an upper boundary edge 66 near the proximal open end 15 and/or near the elbow, signifying that they are in the yellow level of the program. During their first class they would be introduced to the requirements of the yellow level and the skills they will need to learn to progress. Each of these requirements are represented with a primary activity badge or Skill Badge that they can earn once mastering the corresponding skill.

The participant would also be introduced to extra-curricular or secondary activity badges that denote accomplishments for actions that are not based on skill growth. For example, each week there may be a focus on a behavioral or “character theme” such as sharing, caring, or cooperation. Thus, a student or participant may earn a Character Badge for exhibiting behavior that is in keeping with the character theme of the week, but completely independent of their skill acquisition progress.

Another secondary activity badge may be an Attendance Badge, which is given out to any participant who has attended the class that day. The main purpose of this badge is as a form of accountability and data tracking. If a student or participant has accumulated a number of Attendance Badges but very few Skill Badges or Character Badges, then the administrators, teachers and parents will recognize that adjustments need to be made inside the program or for the individual student or participant.

In an exemplary program, a teacher may carry Badges with them during the class, either in their pockets or a small pack they wear. If a child displays a mastery of a skill, or exhibits a behavior in keeping with the character theme of the week, then the teacher may pull the appropriate Badge from their pocket or pack, peel the backing off the Badge, and adhere the Badge to the Sleeve in the appropriate location and orientation. The teacher then asks the class to acknowledge the accomplishment of the student with a round of applause. The class then returns their previous activity.

It is the hope of the program that when a participant receives a Badge on their Achieve Sleeve during class it will generate a positive feeling of accomplishment in the recipient while simultaneously motivating classmates to earn their own badges. There are clear advantages to utilizing an instantaneous and permanent adhesion process for the Badges: 1) The participant receives a tangible, physical symbol at the very moment of their accomplishment, and 2) There is no added work or steps needed, such as sewing or ironing, in order for the Badge to be attached to the Sleeve, creating convenience for the participant or parent of the participant.

The foundation or reference point badge 12 according to the presently disclosed subject matter is generally depicted and referenced at 12. The foundation or reference point badge 12 is preferably (adhesively) attachable to the outer sleeve surface 20 at the posterior side 18 and adjacent the proximal open end 15 as generally depicted in FIGS. 1, 5-10, and 12. The foundation or reference point badge 12 preferably comprises a straight proximal foundation badge edge as at 21 preferably positionable in parallel relation an upper boundary edge 66 of the sleeve 11; an arcuate distal foundation badge edge as at 22; an inner foundation badge side 23; and an outer foundation badge side as at 24.

The inner foundation badge side 23 is preferably outfitted or provided with an adhesive layer as at 25. The outer foundation badge side 24 at the distal foundation badge edge 22 comprises an array of primary activity badge alignment demarcations as at 26. The array of primary activity badge alignment demarcations 26 are preferably aligned end-to-end along the distal foundation badge edge 22 for demarcating an array of activity badge alignment regions 27 in distal or outer adjacency to the distal foundation badge edge 21.

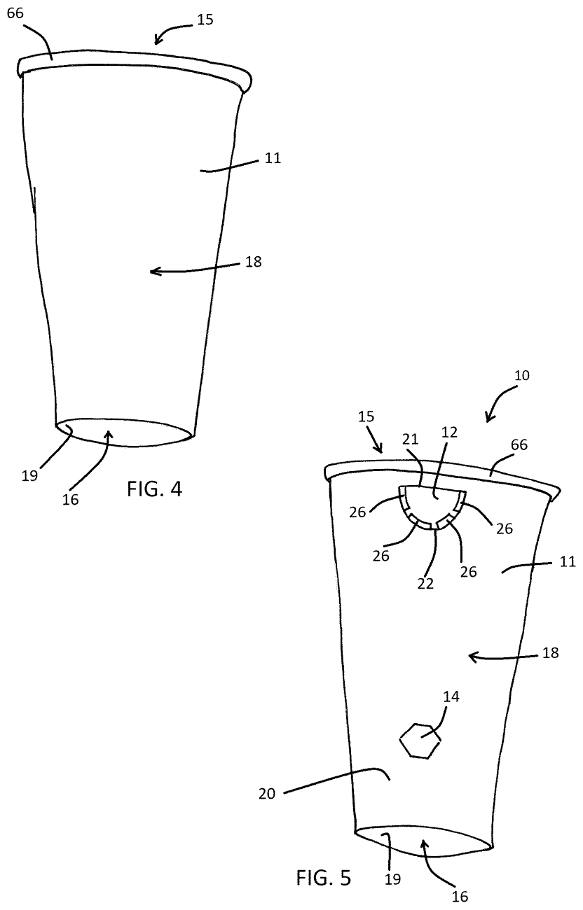

Comparatively referencing FIGS. 1-10, the reader will there consider that the activity or behavior reinforcement badges according to the presently disclosed subject matter may be (adhesively) attached to the outer sleeve surface 20 in a sequential manner so as to reinforce the various activities or behaviors symbolized by the various badges for behavior reinforcement. In other words, as challenges 82, drills 81, and goals 80 are completed for any given primary activity as set forth in the curricula 92, a primary activity badge 13 may be attached to the sleeve 11 in a preferred sequential manner so that the user or participant can display his or her achievements such that a record of achievement is displayed and continually built upon the sleeve 11 in a sequential progression as particularly and comparatively depicted in FIGS. 4-10.

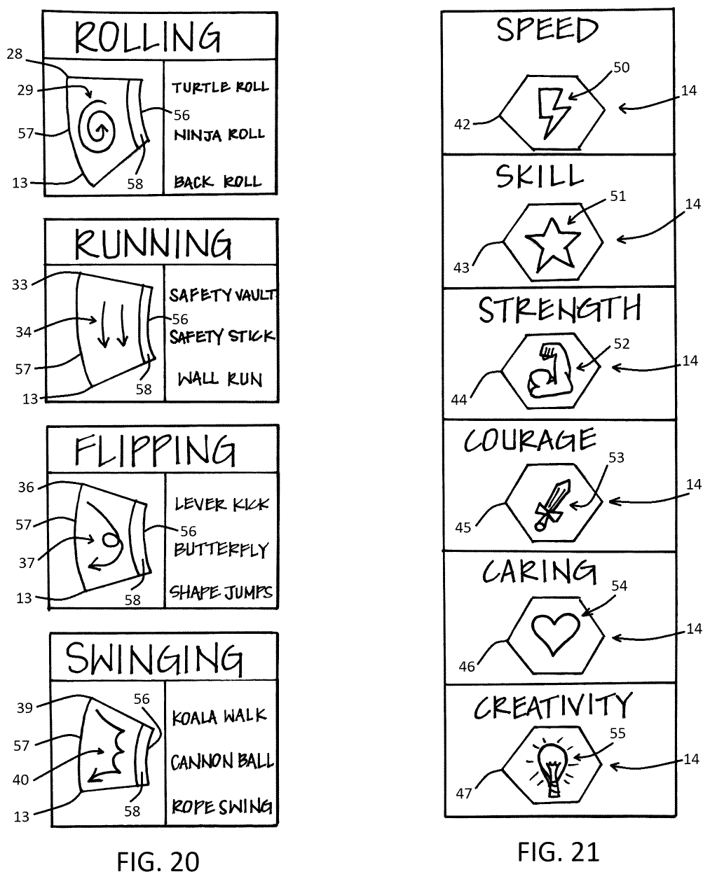

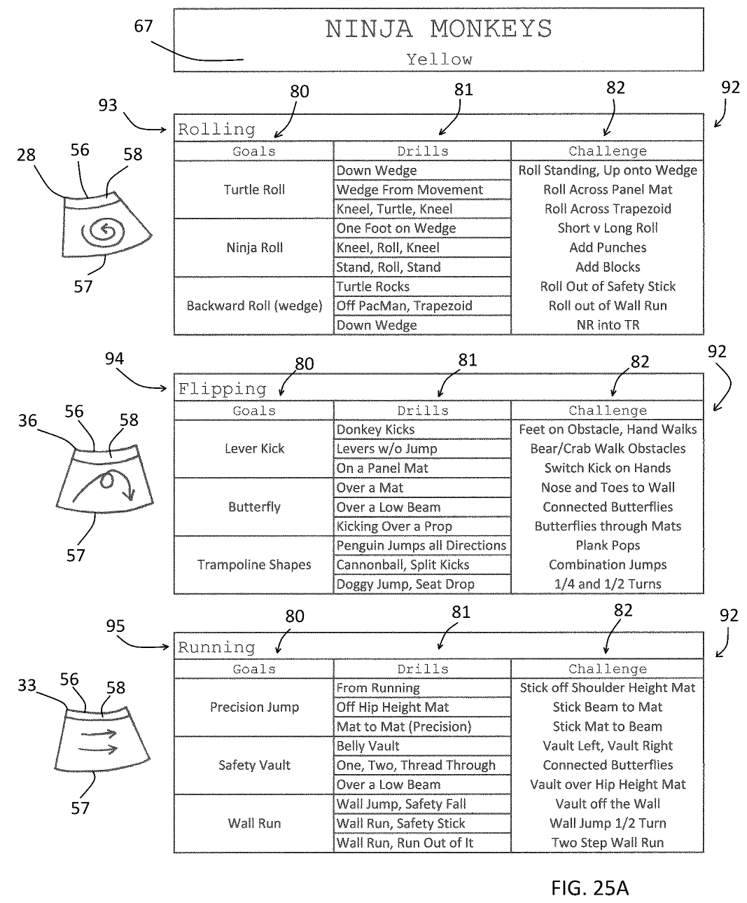

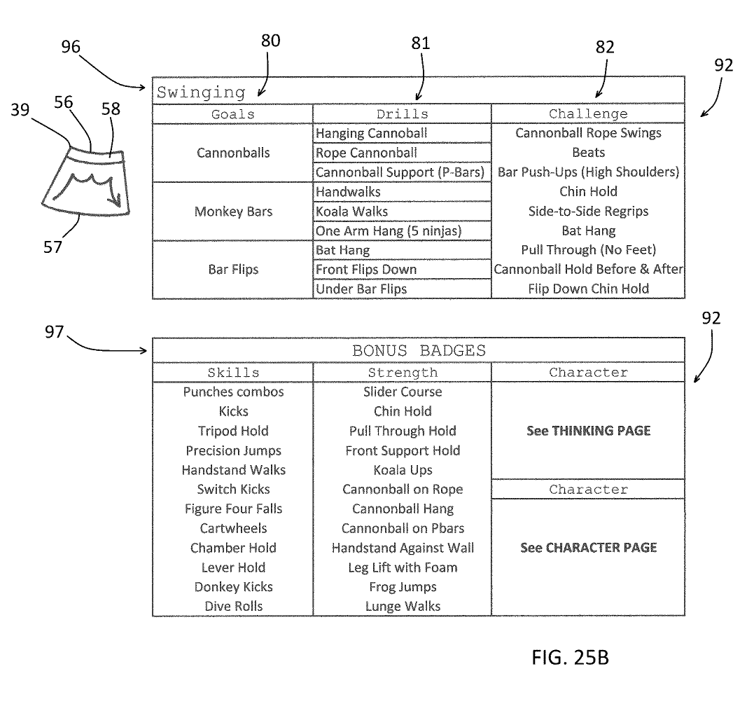

Comparatively referencing FIGS. 20 and 25A, the reader will there consider the “Rolling” type primary activity badge 28 may be achieved once the user demonstrates a certain mastery of the goals 80, drills 81, and challenges 82 as specified in the curriculum 92 for the rolling activity or skill set 93. The “Rolling” type primary badge 28 depicts a rolling symbol 29 upon an outer primary activity badge side 30. Once the user demonstrates a certain mastery of the goals 80, drills 81, and challenges 82 specified for the rolling skill set 93, the adhesive layer 25 of the inner primary activity badge side 31 may be adhesively attached to the activity badge alignment region 27 set aside for the “Rolling” type primary activity badge 28 as depicted and referenced at 32 in FIG. 1A.

Comparatively referencing FIGS. 20 and 25A, the reader will there consider the “Running” type primary activity badge 33 may be achieved once the user demonstrates a certain mastery of the goals 80, drills 81, and challenges 82 specified in the curriculum 92 for the running activity or skill set 95. The “Running” type primary badge 33 depicts a running symbol 34 upon the outer primary activity badge side 30. Once the user demonstrates a certain mastery of the goals 80, drills 81, and challenges 82 specified for the running skill set 95, the adhesive layer 25 of the inner primary activity badge side 31 may be adhesively attached to the activity badge alignment region 27 set aside for the “Running” type primary activity badge 33 as depicted and referenced at 35 in FIG. 1A.

Comparatively referencing FIGS. 20 and 25A, the reader will there consider the “Flipping” type primary activity badge 36 may be achieved once the user demonstrates a certain mastery of the goals 80, drills 81, and challenges 82 specified in the curriculum 92 for the flipping activity or skill set 94. The “Flipping” type primary badge 36 depicts a flipping symbol 37 upon the outer primary activity badge side 30. Once the user demonstrates a certain mastery of the goals 80, drills 81, and challenges 82 specified in the curriculum 92 for the flipping activity or skill set 94, the adhesive layer 25 of the inner primary activity badge side 31 may be adhesively attached to the activity badge alignment region 27 set aside for the “Flipping” type primary activity badge 36 as depicted and referenced at 38 in FIG. 1A.

Comparatively referencing FIGS. 20 and 25B, the reader will there consider the “Swinging” type primary activity badge 39 may be achieved once the user demonstrates a certain mastery of the goals 80, drills 81, and challenges 82 specified in the curriculum 92 for the swinging activity or skill set 96. The “Swinging” type primary badge 39 depicts a swinging symbol 40 upon the outer primary activity badge side 30. Once the user demonstrates a certain mastery of the goals 80, drills 81, and challenges 82 specified in the curriculum 92 for the swinging skill set 96, the adhesive layer 25 of the inner primary activity badge side 31 may be adhesively attached to the activity badge alignment region 27 set aside for the “Swinging” type primary activity badge 39 as depicted and referenced at 41 in FIG. 1A.

The behavior enhancement and goal-setting sleeve arrangement 10 according to the presently disclosed subject matter thus contemplates the use of at least one primary activity badge 13. The primary activity badge(s) 13 are attachable to the outer sleeve surface 20 at the array of activity badge alignment regions 27 in distal or outer adjacency to the distal foundation badge edge 22. Each primary activity badge 13 preferably comprises a proximal primary activity badge edge as at 56 and a distal primary activity badge edge as at 57. Each primary activity badge 13 further preferably comprises a foundation badge alignment demarcation 58 at the proximal primary activity badge edge 56. The foundation badge alignment demarcations 58 aid the user in alignment of the respective primary activity badges 13 with the foundation or reference point badge 12 adjacent the distal foundation badge edge 22.

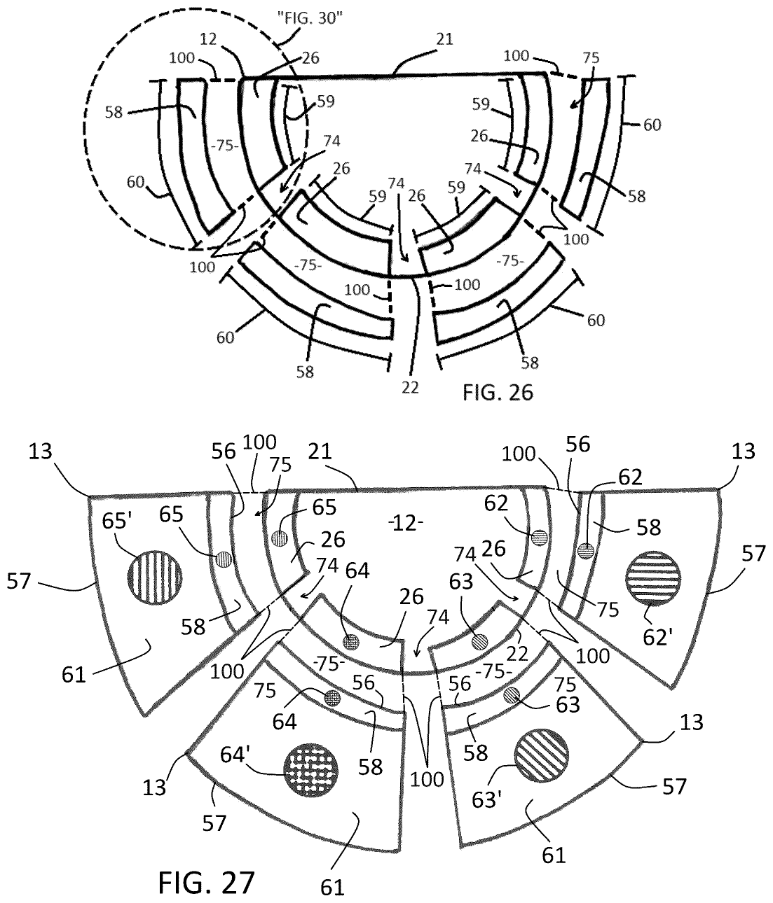

It will be recalled that the distal foundation badge edge 22 is preferably arcuate in form or comprises a distal foundation badge edge arc length. The array of primary activity badge alignment demarcations 26 are further preferably arcuate in form or preferably comprise foundation badge demarcation arc lengths as at 59. The arcuate foundation badge alignment demarcations 58 at the proximal primary activity badge edges 56 preferably comprise a primary activity badge demarcation arc length as at 60, which primary activity badge demarcation arc lengths 60 are preferably greater in magnitude than the foundation badge demarcation arc lengths 59 for aiding the user in spacing and aligning primary activity badges 13 in spaced relation to the arcuate or non-linear distal foundation badge edge 22 of the foundation or reference point badge 12 as generally depicted in FIG. 26.

Referencing FIGS. 26 through 30, the reader will there further consider the primary activity badge alignment demarcations 26 are arcuately aligned in end-to-end relation along the arcuate or non-linear distal foundation badge edge 22 wherein neighboring primary activity badge alignment demarcations 26 are aligned opposite a foundation badge gap 74 such that linear, laterally opposed first demarcation edges 70 laterally define the foundation badge gaps 74 and extend along edge planes as at 100.

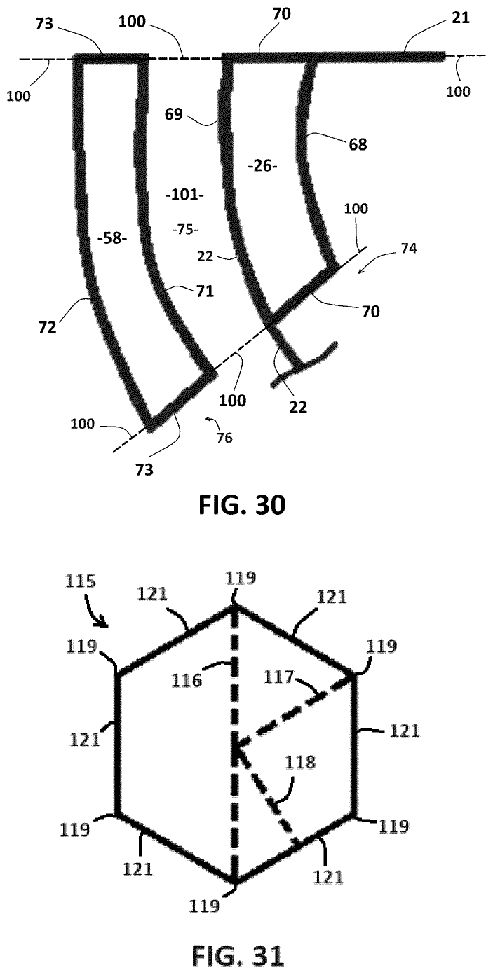

In other words, the foundation edge gaps 74 are spaced intermediate the array of primary activity badge alignment demarcations 26. Specifically referencing FIG. 30, the reader will there see the primary activity badge alignment demarcations 26 each preferably comprise an arcuate or non-linear, proximal primary activity badge alignment demarcation edge as at 68; an arcuate or non-linear, distal primary activity badge alignment demarcation edge as at 69; and linear, laterally opposed first demarcation edges as at 70. The linear, opposed first demarcation edges 70 define opposed sides of the foundation badge gaps 74 along the edge planes 100.

The foundation badge alignment demarcations 58 each preferably comprise an arcuate or non-linear, proximal foundation badge alignment demarcation edge as at 71 (coextensive with the proximal primary activity badge edge 56); an arcuate or non-linear, distal foundation badge alignment demarcation edge as at 72; and linear, laterally opposed second demarcation edges as at 73. The foundation badge alignment demarcations 58 are attached to the array of activity badge alignment regions 27 in distally spaced adjacency (as at spacing 101) to the arcuate or non-linear distal foundation badge edge 22 such that the linear, laterally opposed first demarcation edges 70 provide visual cues for aligning the linear, laterally opposed second demarcation edges 73 of the primary activity badges 13 along the edge planes 100.

In other words, the linear, opposed first demarcation edges 73 of each primary activity badge 13 are alignable along corresponding edge planes 100 for guiding placement of the primary activity badges 13 at the array of activity badge alignment regions 58 such that the edge planes 100 enable the user to properly (a) distance the primary activity badges 13 in gapped relation relative to the arcuate distal foundation badge edge 22 as at gaps 75 and (b) align the primary activity badges 13 in gapped relation (i) to one another as at gaps 76 and (ii) the foundation badge 12. The end result is that the primary activity badges 13 are (i) spaced in gapped relation to one another as at gaps 76 defined by the edge planes 100 and (ii) spaced in gapped relation relative to the foundation badge 12 in distal adjacency to the arcuate distal foundation badge edge 22 as at gaps 75.

It will be further noted that the array of arcuate or non-linear primary activity badge alignment demarcations 26 preferably comprise primary activity badge demarcation-matching coloration and that the foundation badge alignment demarcations 58 comprise foundation badge demarcation-matching coloration. In this regard, an exemplary coloration scheme or layout is generally depicted in FIG. 27. Referencing FIG. 27, the reader will there consider the primary activity badges 13 in clockwise manner from right-to-left, a first primary activity badge 13 comprising blue coloration depicted at hatch marking 62 upon both the arcuate or non-linear foundation badge alignment demarcation 58 and upon the symbol-bearing region 61 distal to the arcuate or non-linear foundation badge alignment demarcation 58. The symbol-bearing region bears a blue hatch marking referenced at 62′. The blue coloration as at hatch marking 62 on the arcuate or non-linear foundation badge alignment demarcation 58 matches the blue coloration as at hatch marking 62 born upon the corresponding arcuate or non-linear primary activity badge alignment demarcation 26. The demarcation-matching coloration aids the user in aligning the primary activity badges 13 in adjacency to the foundation badge 12 via a color-matched badge alignment demarcation pairing.

The reader will note the different sizes in hatch markings 62 and 62′. The difference in sizes between match markings 62 and 62′ denotes different shades of blue as practiced in the preferred embodiment. The behavior enhancement and goal-setting sleeve arrangement 10 according to the presently disclosed subject matter contemplates a color-matched badge alignment demarcation pairing as at color matched hatch markings 62 and further contemplates a first color shade as at 62 whereby the primary activity badges 13 each further preferably comprise a second color shade as at 62′ distal to the foundation badge alignment demarcation 58 within the symbol-bearing regions 61 of the primary activity badges 13.

A second primary activity badge 13 comprising green coloration is depicted at hatch marking 63 upon both the foundation badge alignment demarcation 58 and upon the symbol-bearing region 61 distal to the foundation badge alignment demarcation 58 and referenced at 63′. The green coloration as at hatch marking 63 on foundation badge alignment demarcation 58 matches the green coloration as at hatch marking 63 born upon the corresponding primary activity badge alignment demarcation 26. The reader will note the different sizes in hatch markings 63 and 63′. The difference in sizes between match markings 63 and 63′ denotes different shades of green as practiced in the preferred embodiment in keeping with the foregoing descriptions.

A third primary activity badge 13 comprising yellow coloration is depicted at hatch marking 64 upon both the foundation badge alignment demarcation 58 and upon the symbol-bearing region 61 distal to the foundation badge alignment demarcation 58 and referenced at 64′. The yellow coloration as at hatch marking 64 on foundation badge alignment demarcation 58 matches the yellow coloration as at hatch marking 64 born upon the corresponding primary activity badge alignment demarcation 26. The reader will note the different sizes in hatch markings 64 and 64′. The difference in sizes between match markings 64 and 64′ denotes different shades of yellow as practiced in the preferred embodiment in keeping with the foregoing descriptions.

A fourth primary activity badge 13 comprising red coloration is depicted at hatch marking 65 upon both the foundation badge alignment demarcation 58 and upon the symbol-bearing region 61 distal to the foundation badge alignment demarcation 58 and referenced at 65′. The red coloration as at hatch marking 65 on foundation badge alignment demarcation 58 matches the red coloration as at hatch marking 65 born upon the corresponding primary activity badge alignment demarcation 26. The reader will note the different sizes in hatch markings 65 and 65′. The difference in sizes between match markings 65 and 65′ denotes different shades of red as practiced in the preferred embodiment in keeping with the foregoing descriptions.

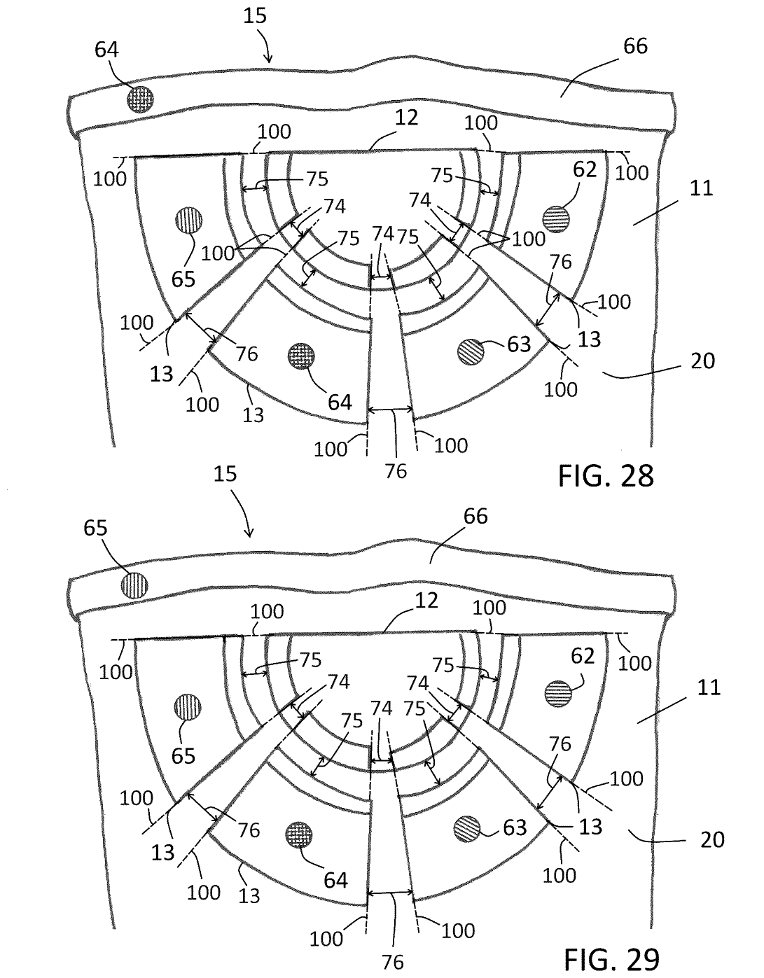

Comparatively referencing FIG. 28 versus FIG. 29, the reader will there note that the sleeve 11 may preferably comprise a color-coded upper boundary edge 66 adjacent the proximal open end 15. FIG. 28 depicts a color-coded upper boundary edge 66 outfitted with yellow coloration as at hatch marking 64. In this regard, the reader will further comparatively reference FIG. 25A, and the header 67 of the set of curricula 92 there depicted denoting a “yellow” class. As participants complete the curricula 92 of the “yellow” class, the participants graduate to a new set of curricula 92 and when entering a new set of curricula 92, a new behavior enhancement and goal-setting sleeve arrangement 10 may be preferably provided as outfitted with a new color-coded upper boundary edge 66 of differing coloration.

A “red” class is denoted by hatch marking 65 upon the upper boundary edge 66 in FIG. 29. The behavior enhancement and goal-setting sleeve arrangement 10 according to the presently disclosed subject matter may provide the basis for a behavior enhancement and goal-setting system comprising an array of behavior enhancement and goal-setting apparatuses 10, each of which may bear a color-coded upper boundary edge 66. The behavior enhancement and goal-setting system according to the presently disclosed subject matter may thus be said to comprise a first sleeve 11 and at least one second sleeve 11 substantially identical to the first sleeve 11 but for a differently colored upper boundary edge 66 or differently colored portion of the sleeve(s) 11. The first sleeve 11 comprises a first color-coded upper boundary edge 66 or portion at the proximal open end 15, the first color-coded upper boundary edge 66 denotes a first set of curricula 92 associated with a first color (e.g. a “yellow” class). The second sleeve 11 in the series may comprises a second color-coded upper boundary edge 66 or portion at the proximal open end 15 for denoting a second set of curricula 92 associated with a second color (e.g. a “red” class). The second sleeve 11 may be awarded to the user upon the user's completion of the first set of curricula 92.

Referencing FIG. 25B, the reader will there consider a “bonus badges” portion 97 portion of the curricula 92. The bonus badges portion 97 of the curricula 92 lists certain other secondary skills and attributes of the curricula 92 that contribute to positive behavior development within the curricula 92 such as speed, skill, strength, courage, caring, and creativity. Bonus badges may be awarded when behaviors within the respective categories are witnessed by the development team or coaches within the activity setting. The behavior enhancement and goal-setting sleeve arrangement 10 according to the presently disclosed subject matter thus contemplates the inclusion of at least one secondary activity badge 14, which secondary activity badge(s) comprise an outer secondary activity badge side 48 and an inner secondary activity badge side 49 with an adhesive layer 25. The adhesive layer 25 of the inner secondary activity badge side 49 is preferably attachable to the outer sleeve surface 20 in distal adjacency to the array of activity badge alignment regions 27 at the posterior side 18 in proximal adjacency to the distal open end 16.

A speed type bonus badge or secondary activity badge 14 is generally depicted and referenced at 42 having a speed type symbol 50 thereupon; a skill type bonus badge or secondary activity badge 14 is generally depicted and referenced at 43 having a skill type symbol 51 thereupon; a strength type bonus badge or secondary activity badge 14 is generally depicted and referenced at 44 having a strength type symbol 52 thereupon; a courage type bonus badge or secondary activity badge 14 is generally depicted and referenced at 45 having a courage type symbol 53 thereupon; a caring type bonus badge or secondary activity badge 14 is generally depicted and referenced at 46 having a caring type symbol 54 thereupon; and a creativity type bonus badge or secondary activity badge 14 is generally depicted and referenced at 47 having a creativity type symbol 55 thereupon.

The behavior enhancement and goal-setting system and sleeve arrangements 10/110 according to the presently disclosed subject matter essentially provides a user with a wearable physical representation of the user's progress in an activity so as to reinforce behavior supportive of the activity. In some applications, the behavior enhancement and goal-setting system comprises at least one wearable fabric article and at least one activity reinforcement badge. In some embodiments, the wearable fabric article may be characterized by a sleeve 11.

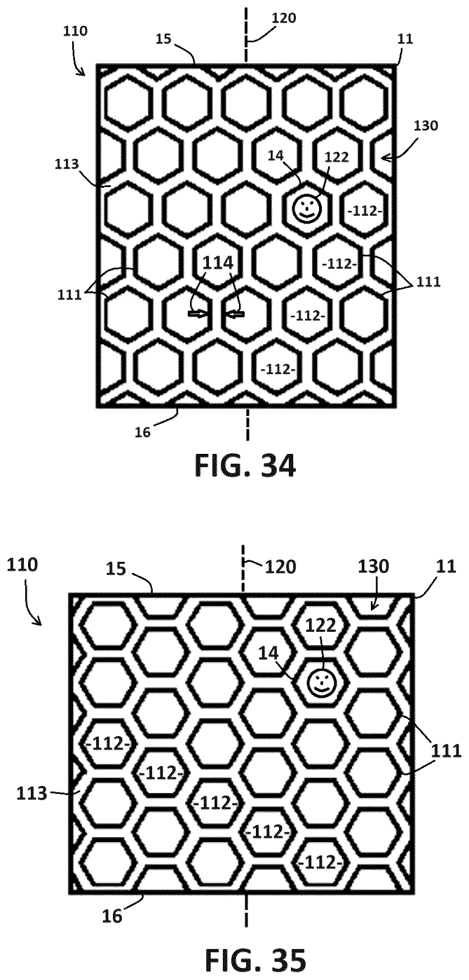

The wearable fabric article has an outer article or sleeve surface as at 20. In some embodiments, the outer article or sleeve surface 20 is marked with a tessellated pattern or periodic tiling pattern as at 130 in FIGS. 34-39. The periodic tiling pattern 130 comprising an array of tiles 111 configured to visually demarcate an array of activity reinforcement badge attachment sites 112. The activity reinforcement badge, as exemplified by benchmark badge 14, is configured for attachment to a select activity reinforcement badge attachment site of the array of activity reinforcement badge attachment sites 112. Each activity reinforcement badge is sized and shaped to correspond to a tile size and a tile shape within the array of tiles 111.

In some embodiments, the periodic tiling pattern is a regular periodic tiling pattern comprising an array of regular shapes. In some embodiments, the array of regular shapes is defined by tile-defining grid pattern 113. The tile-defining grid pattern 113 comprises a uniform tile-to-tile thickness 114 in some embodiments. In some embodiments, the regular periodic tiling pattern is a hexagonal tiling pattern. In some embodiments, the hexagonal tiling pattern comprises an array of regular hexagonal tiles 115 or shapes each of which comprise a long diagonal axis 116, and edge length 117, and an incircle radius 118 as generally depicted and referenced in FIG. 31.

The long diagonal axes 116 intersect opposed oblique angles as the vertices 119. The edge length 117 is equal to the length of every edge 121. This is the same as the circumcircle radius (the radius of a circle circumscribing the hexagon). In other words, if the hexagon were to be drawing such that the vertices 119 were positioned within the circumference of a circle, the edge length 117 is the radius of the circumcircle. The incircle radius 118 is the radius of a circle that fits inside the hexagon.

In some embodiments, the long diagonal axes 115 extend lengthwise across the outer article surface 20 transverse to the proximal open end 15 and distal open end 16 and parallel to the sleeve long axis 120 as generally depicted in FIG. 34. In these embodiments, the long diagonal axis 116 extends medially through left and right halves of a character image 122 printed upon the badge 14. In some embodiments, the long diagonal axes 115 extend widthwise across the outer article surface 20 in parallel relation to the proximal open end 15 and distal open end 16 transverse to the sleeve long axis 120 as generally depicted in FIGS. 35-39. In these embodiments, the long diagonal axis 116 extends through upper and lower halves of the character image 122. The sleeve 11, which can be made of natural or synthetic fabrics, has a pattern of specifically proportioned hexagonal tiles 115 printed onto the fabric which serve as the placement indicator for adhesive based activity badges to be affixed, in some embodiments.

To maximize the amount of benchmark badges affixed to any clothing or accessory, a hexagon shape with a honeycomb placement pattern has been found to create the highest level of effectiveness. In particular, by rounding the corners of the hexagon badges the adhesive performs at its best due to its lack of sharp corners which can snag or catch on environmental objects during physical activities. In addition, the honeycomb placement of badges 14 creates the least amount of wasted space on any clothing item or worn accessory. It is noted hexagonal tiling creates a partition with equal-sized cells, while minimizing the total perimeter of the cells. Known in geometry as the “honeycomb conjecture”.

A hexagonal tile 115 basis, as further tessellated, provides the greatest number of tiles within a fixed surface area for the outer article surface 20. The benchmark badges and sleeve 11 can both be customized to fit the goals of any particular business or program. This is done be creating graphic overlays on top of the honeycombed or tessellated hexagonal, badge placement pattern. Additionally, any business or program can create badge images to match their specific brand and benchmarks that have been uniquely constructed for their business. They can also determine the placement order of the badges, the placement location, and the meaning behind each individual image or pattern.

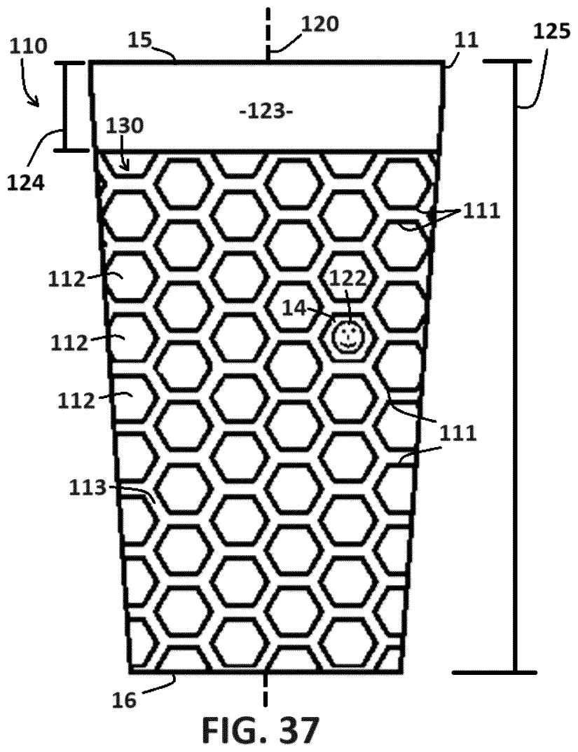

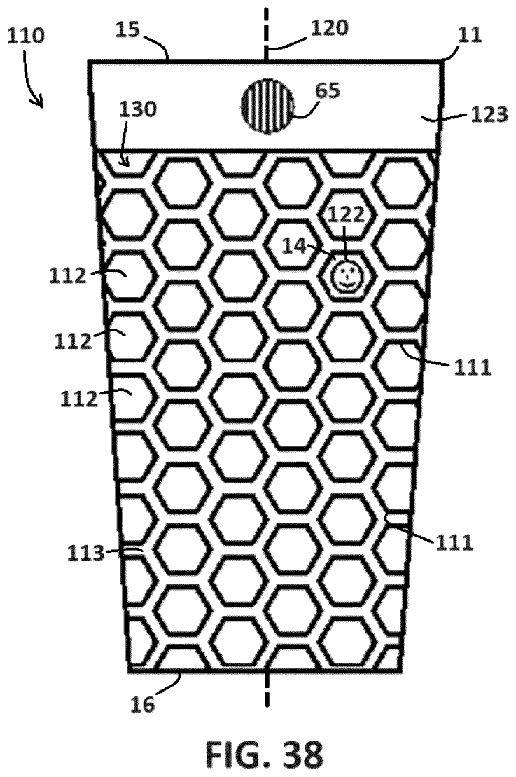

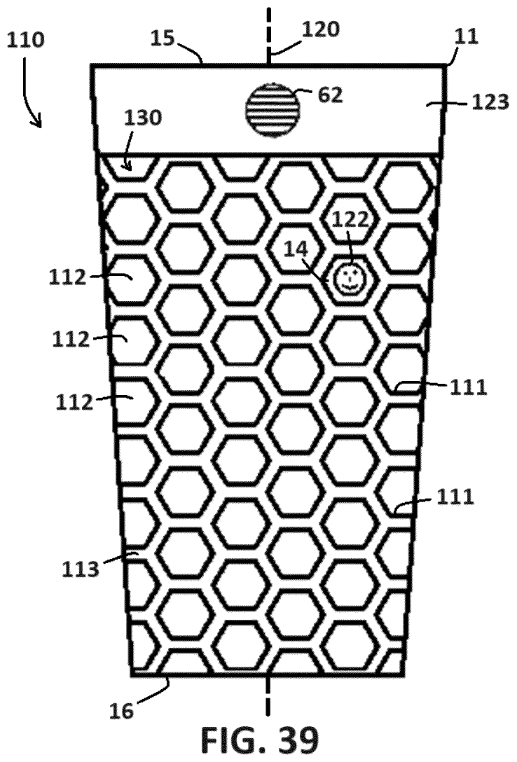

In some embodiments, the outer article or sleeve surface comprises a band portion 123. In some embodiments, the band portion 123 is proximally located, distally extending from the proximal open end 15. In some embodiments, the band width 124 is less than half a sleeve length 125 of the sleeve 11 as generally depicted and referenced in FIG. 37. In some embodiments, the band portion 123 may be characterized by being color coded or as a color code band portion. Comparatively referencing FIGS. 38 and 39, the reader will there consider hatch markings 65 and 62. As earlier described, the hatch marking 65 denotes red coloration and the hatch marking 62 denotes blue coloration. In some embodiments, the color code band portion 123 may comprise a color code as exemplified by red coloration hatch marking 65 or blue coloration hatch marking 62 for denoting a participant activity level.

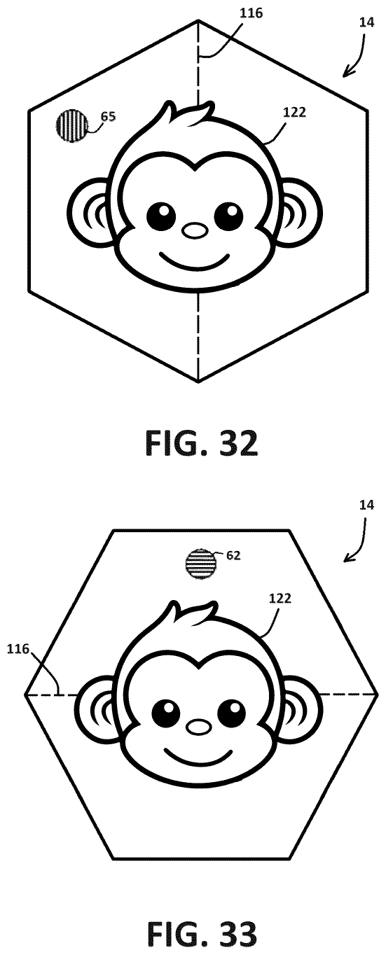

In some embodiments, the activity reinforcement badge(s) may comprise a color code badge portion corresponding to the color code band portion 123. Comparatively referencing FIG. 32 and FIG. 38, for example, it will there be seen that the badge 14 also comprises red coloration hatch marking 65. Similarly comparing FIG. 33 and FIG. 39, it will there be seen that the badge 14 also comprises blue coloration hatch marking 62. Accordingly, in some embodiments, the badges 14 may comprise certain surface coloration that correspond to the color of the band portion 123 for further categorizing rewarded behavior with in the behavior enhancement and goal-setting system based on sleeve arrangements 10/110.

The behavior enhancement and goal-setting system according to the presently disclosed subject matter may comprise a first sleeve 11 and at least one second sleeve 11 such that the first sleeve comprises a first color code band portion 123. The first color code band portion 65/123 denote a first set of curricula associated with a first color as at red coloration hatch marking 65. The at least one second sleeve 11 comprises a second color code band portion 123. The second color code band portion 123 denotes a second set of curricula associated with a second color as a blue coloration hatch marking 62. The at least one second sleeve 11 may be awarded to the user upon the user's completion of the first set of curricula.

While the above descriptions contain much specificity, this specificity should not be construed as limitations on the scope of the presently disclosed subject matter, but rather as an exemplification thereof. The intended application of the presently disclosed subject matter is for any program-based service that utilizes a curriculum or structured experience (i.e.: a school, theme park, or lesson-based private business) in which benchmarks are achieved. Those benchmarks are then recognized or rewarded with a badge that is then affixed to an article of clothing or worn accessory, such as a hat, backpack, or wristband.

Specifically, a badge created with the unique layering process of a printable fabric, a fabric adhesive and paper backer. Additionally, the affixing process is specifically performed by a person peeling the fabric badge off of its backer and then placing the badge onto the clothing or accessory in a predetermined arrangement that is in alignment with the curriculum or structured experience created by the program-based service.

In some embodiments, the sleeve 11 is illustrated as beginning at the wrist and completing just short of the elbow or is preferably donned upon the entire length of a participant's forearm. However, the sleeve can be any size on the forearm in accordance with the desire of the company or program utilizing the product. Should the purchaser choose to have a shorter sleeve that only partially covers the forearm, or longer so that it extends past the elbow to just below the deltoid, those choices would have no bearing on the functionality of the product.

The sleeve also functions equally well on the lower leg or calf or thigh muscle. With some simple adjustments a client could place the product on either of those areas of the body and it would still be appropriate for rigorous activity and successfully display the activity reinforcement badges in keeping with the behavior modification aspects of the presently disclosed subject matter. Moreover, the sleeve 11 may be attached to another form-fitting product, such as a compression sleeve or rash guard shirt, and still function according to the design and application of the product. This adjustment would allow the wearer the dual benefit of whatever the secondary product, such as the aforementioned compression sleeve, and the activity reinforcement badges or Achieve Sleeve Badges.

The sleeve 11 is preferably formed from form-fitting fabric material having certain elasticity, and preferably comprises a proximal open end, a distal open end, an anterior side, a posterior side, a sleeve axis, an inner sleeve surface, and an outer sleeve surface. The outer sleeve surface primarily receives activity reinforcement badges, and inner sleeve surfacing primarily opposes or engages a user's anatomy or skin surface exemplified by a user's forearm or lower leg as alternatively outfitted with other structures such as a compression sleeve.

While it is the intent of the designer to use the Badges on the Sleeve, as noted, it must be acknowledged that the application of the Badges can and will function on any fabric similar to that of the Sleeve. And, so, the application opportunities for the product can be extended to any product meeting the fabric criteria for the adhesive, meaning any clothing or accessory created with cotton or blended material. For example, a person or entity could design, print, and cut the Badge product and then adhere it to a cotton T-shirt for the purpose of acknowledging the completion of any activity created by said person or entity.

The Badges can also be designed with the human body's unique shaping in mind. For example: A badge could be designed and cut in order to fit precisely around the wrist, or to follow the contours of the forearm in a way that accentuates the anatomy. If the product were to extend past the elbow, or the knee, a Badge, or multiple badges, could be created to fit appropriately around the joint in a safe and desirable fashion. All of this can be done while simultaneously achieving the product goals.

The activity reinforcement badges according to the presently disclosed subject matter, including the foundation or reference point badge 12, the primary activity badges 13, and the secondary activity badges 14, are preferably adhesively attachable to the outer sleeve surface, but could conceivably also be attached to the outer sleeve surface in any number of ways, including permanent stitching and hook and loop fastening means.

Accordingly, although the behavior enhancement and goal-setting system and sleeve arrangements, including the badge system for outfitting any article of clothing, according to the presently disclosed subject matter have been described by reference to a number of different embodiments, aspects, and features, it is not intended that the novel combinations or assemblies be limited thereby, but that modifications thereof are intended to be included as falling within the broad scope and spirit of the foregoing disclosure, the appended drawings, and the following claims.

What is claimed is:

1. A behavior enhancement and goal-setting system for providing a user with a wearable physical representation of the user's progress in an activity so as to reinforce behavior supportive of the activity, the behavior enhancement and goal-setting system comprising:
at least one wearable fabric article having an outer article surface, the outer article surface being marked with a periodic tiling pattern comprising an array of tiles configured to visually demarcate an array of activity reinforcement badge attachment sites; and
at least one activity reinforcement badge configured for attachment to a select activity reinforcement badge attachment site of the array of activity reinforcement badge attachment sites, the at least one activity reinforcement badge being sized and shaped to correspond to a tile size and a tile shape within the array of tiles.

2. The behavior enhancement and goal-setting system according to claim 1, wherein the wearable fabric article is characterized by a sleeve, the sleeve comprising a proximal open end, a distal open end, an outer sleeve surface defining the outer article surface and a sleeve length extending along a sleeve long axis.

3. The behavior enhancement and goal-setting system according to claim 1, wherein the periodic tiling pattern is a regular periodic tiling pattern comprising an array of regular shapes.

4. The behavior enhancement and goal-setting system according to claim 2, wherein the array of regular shapes is defined by shape-defining grid pattern, the shape-defining grid pattern comprising a uniform tile-to-tile thickness.

5. The behavior enhancement and goal-setting system according to claim 2, wherein the regular periodic tiling pattern is a hexagonal tiling pattern.

6. The behavior enhancement and goal-setting system according to claim 4, wherein the hexagonal tiling pattern comprises an array of regular hexagonal shapes each of which comprise a long diagonal axis, the long diagonal axes extending widthwise across the outer article surface in parallel relation to the proximal open end and distal open end transverse to the sleeve long axis.

7. The behavior enhancement and goal-setting system according to claim 1, wherein the outer article surface comprises a color code band portion, the color code band portion comprising a color code for denoting a participant activity level.

8. The behavior enhancement and goal-setting system according to claim 6, wherein the at least one activity reinforcement badge comprises a color code badge portion corresponding to the color code band portion.

9. The behavior enhancement and goal-setting system according to claim 1, wherein the color code band portion is proximally located, distally extending from the proximal open end a band thickness.

10. The behavior enhancement and goal-setting system according to claim 6 comprising a first sleeve and at least one second sleeve, the first sleeve comprising a first color code band portion, the first color code band portion for denoting a first set of curricula associated with a first color, the at least one second sleeve comprising a second color code band portion, the second color code band portion for denoting a second set of curricula associated with a second color, the at least one second sleeve being awarded to the user upon the user's completion of the first set of curricula.

11. A behavior enhancement and goal-setting sleeve arrangement for providing a user with a wearable physical representation of the user's progress in an activity so as to reinforce behavior supportive of the activity, the behavior enhancement and goal-setting sleeve arrangement comprising:
a sleeve formed from a fabric material and comprising a proximal open end, a distal open end, an outer sleeve surface and a sleeve length extending along a sleeve long axis;
the outer sleeve surface being marked with a periodic tiling pattern comprising an array of tiles configured to visually demarcate an array of activity reinforcement badge attachment sites; and
at least one activity reinforcement badge configured for attachment to a select activity reinforcement badge attachment site of the array of activity reinforcement badge attachment sites, the at least one activity reinforcement badge being sized and shaped to correspond to a tile size and a tile shape within the array of tiles.

12. The behavior enhancement and goal-setting sleeve arrangement according to claim 11, wherein the periodic tiling pattern is a regular periodic tiling pattern comprising an array of regular shapes.

13. The behavior enhancement and goal-setting sleeve arrangement according to claim 12, wherein the array of regular shapes is defined by shape-defining grid pattern, the shape-defining grid pattern comprising a uniform shape-to-shape thickness.

14. The behavior enhancement and goal-setting sleeve arrangement according to claim 12, wherein the regular periodic tiling pattern is a hexagonal tiling pattern.

15. The behavior enhancement and goal-setting sleeve arrangement according to claim 14, wherein the hexagonal tiling pattern comprises an array of regular hexagonal shapes each of which comprise a long diagonal axis, the long diagonal axes extending widthwise across the outer sleeve surface in parallel relation to the proximal open end and distal open end transverse to the sleeve long axis.

16. The behavior enhancement and goal-setting sleeve arrangement according to claim 14, wherein the hexagonal tiling pattern comprises an array of regular hexagonal shapes each of which comprise a long diagonal axis, the long diagonal axes extending lengthwise across the outer sleeve surface in parallel relation to the to the sleeve long axis.

17. The behavior enhancement and goal-setting sleeve arrangement according to claim 11, wherein the outer sleeve surface comprises a color code band portion, the color code band portion comprising a color code for denoting a participant activity level.

18. The behavior enhancement and goal-setting sleeve arrangement according to claim 17, wherein the at least one activity reinforcement badge comprises a color code badge portion corresponding to the color code band portion.

19. The behavior enhancement and goal-setting sleeve arrangement according to claim 11, wherein the color code band portion is proximally located, distally extending from the proximal open end a band thickness.

20. The behavior enhancement and goal-setting sleeve arrangement according to claim 19, wherein the band thickness is less than half a sleeve length.
